(19) United States
(12) Patent Application Publication
(10) Pub. No.: US 2017/0020422 A1
(43) Pub. Date: Jan. 26, 2017

(54) Device for Biosensing With Indwelling Venous Catheter

(71) Applicant: DIAGNOSTIC BIOCHIPS, Glen Burnie, MD (US)

(72) Inventors: Emma Bigelow, Glen Burnie, MD (US); Rohan Pais, Glen Burnie, MD (US); Rob Collins, Glen Burnie, MD (US); Brian Jamieson, Glen Burnie, MD (US)

(73) Assignee: DIAGNOSTIC BIOCHIPS, INC., Glen Burnie, MD (US)

(21) Appl. No.: 15/110,469

(22) PCT Filed: Apr. 8, 2015
(86) PCT No.: PCT/US15/24970

§ 371 Date: Jul. 8, 2016

Related U.S. Application Data
(60) Provisional application No. 61/976,739, filed on Apr. 8, 2014.

(51) Int. Cl. A61B 5/1486; A61M 25/00; A61B 5/00; A61B 5/1473; A61B 5/145

(52) U.S. Cl. CPC A61B 5/14865; A61B 5/1473; A61B 5/14525; A61B 5/6848; A61B 5/6852; A61B 5/14532; A61M 25/0009; B33Y 10/00

(57) ABSTRACT

The specification and drawings show an embodiment of the present invention in the form of a device comprising one or more biosensors either placed or imbedded in a catheter, needle or combination of the two. The catheter or needle comprises exclusionary slits upstream of the biosensor(s), angled so that fluid flows in through the slits and down past the biosensors. The wires connecting the biosensors to external monitoring and/or analytical apparatus are wired through the catheter or needle material. Also provided herein are methods for detection of small molecules using the device described herein.

### BACKGROUND

Currently, sedation is monitored with vital signs and sometimes with brain activity (a BIS monitor—bi-spectral index—measures awareness, but is not very reliable). Without knowing actual blood concentrations of anesthetics, anesthesiologists tend to give a bolus of anesthetics at the beginning of surgery. This sometimes results in patients being under for longer than necessary, which requires the patient to stay in the hospital for longer. Sometimes patients metabolize anesthetics quickly and begin to wake up during the surgery, also not ideal.

Anesthesiologists regularly struggle with dosing decisions because, as they watch blood pressure as an indicator of dosing, it is unclear whether pressure has changed due to change in blood volume (blood loss or transfusion), or if vasodilation or vasoconstriction has occurred. During a surgery, significant blood is lost, which is not calculated (and is incalculable). The anesthesiologist estimates how many units of blood to give, and then watched the blood pressure to decide if blood supply has been adequately replenished. Deciding to dose anesthetics or give more blood based on blood pressure can be the most difficult and common judgment moment for anesthesiologists. This decision is a judgment call because the total circulating blood volume is unknown. Accordingly, it would be a very significant value to know total blood volume at critical moments during surgery.

Other possible applications exist in the ER and ICU, where a time-sensitive measurement of blood volume could add significant value to patient care. Additionally, there is an outpatient test that takes about 1 hour for the diagnosis of chronic fatigue syndrome, anemia, among other blood/RBC disorders. In this test a number of blood samples are collected over time and sent to the lab, resulting in inevitable delays. Patient care would be significantly improved by real-time monitoring.

A typical IV catheter consists of a catheter (small flexible tube) which is placed into a vein using a needle. The catheter forms a sheath around the needle. The needle offers the rigidity and a sharp edge to introduce the catheter into the vein; after which the needle is removed leaving the catheter (which is soft and flexible) in the vein. Using such a catheter, IV fluids can be pumped into the blood.

A biosensor in the blood stream of a subject is subject to a number of forces that could cause it to fail: too rapid flow (not enough time for molecules to stick to biosensor), shear forces, biofouling by clotting factors, non-specific signal due to large proteins that stick to the surface and exclude the target molecule.

### BRIEF DESCRIPTION OF THE DRAWINGS

FIG. 1 shows a side view (A), cut-away (B) and cross-section (C and D) views of one embodiment of a needle or catheter having exclusionary slits and sensors in accordance with the present invention.

FIG. 2 is a side view of a tapered tip needle with built-in biosensor (blue), showing blood flow (red) and buffer flow (yellow).

FIG. 3 is a cross-section view of the needle of FIG. 2.

FIG. 4 is a side view of a needle with retractable biosensor (blue), showing blood flow (red) and buffer flow (yellow).

FIG. 5 shows a needle embodiment, and a cross-section showing possible position of conductors within the needle.

FIG. 6 is a detail view of a Luer lock connector mechanism comprising built-in conductor pads.

### DETAILED DESCRIPTION

Described herein is a biosensor that can be placed in the blood stream while protected from red blood cells, clotting factors, and shear force. This device also allows the flow past the sensor surface to be tuned to the requirements for the binding kinetics of the biosensor. Additionally, as some biosensing elements are degradable in vivo (due to innate immune response/encapsulation, nuclease degradation of aptamers, etc.), this device serves to exclude some of the potential biosensor-degrading elements in physiological samples.

Also described herein is a tool that may be used to continuously monitor the concentrations of various drugs or biomarkers (e.g., chemotherapeutic levels) in the blood using catheter or needle bearing at least one aptamer biosensor as described herein. The catheter or needle is specifically designed to permit prolonged monitoring in blood while avoiding biofouling of the sensor through a boundary layer of buffer flowing past the sensor.

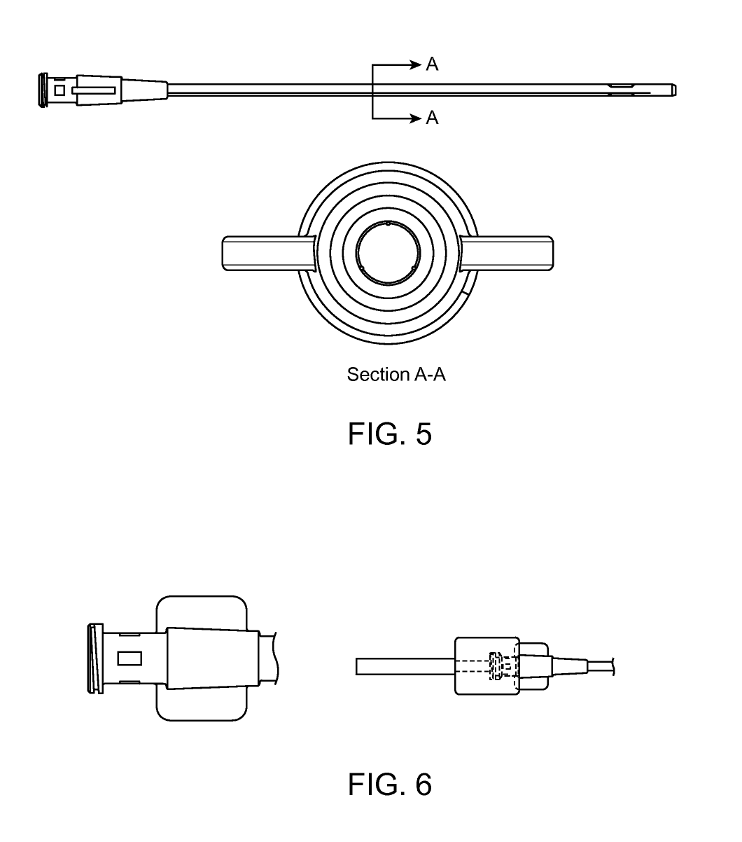

In one embodiment, one or more conductors are placed on the inside of a catheter or needle. These conductors can act as the electrodes for the sensor(s) (e.g., a counter, working and reference electrode). A buffer may be made to flow through the catheter or needle, thereby prolonging the working lifetime of the sensor. There are thin slits in the wall of the catheter or needle, which allow for the diffusion of blood onto the sensor. Such a device is shown in FIG. 5. Other metal contacts located elsewhere in the body besides the catheter may also be used as the counter or reference electrodes for this sensor system.

As used herein, the term “subject” means a human or other organism with a circulatory system into which the device described herein may be inserted.

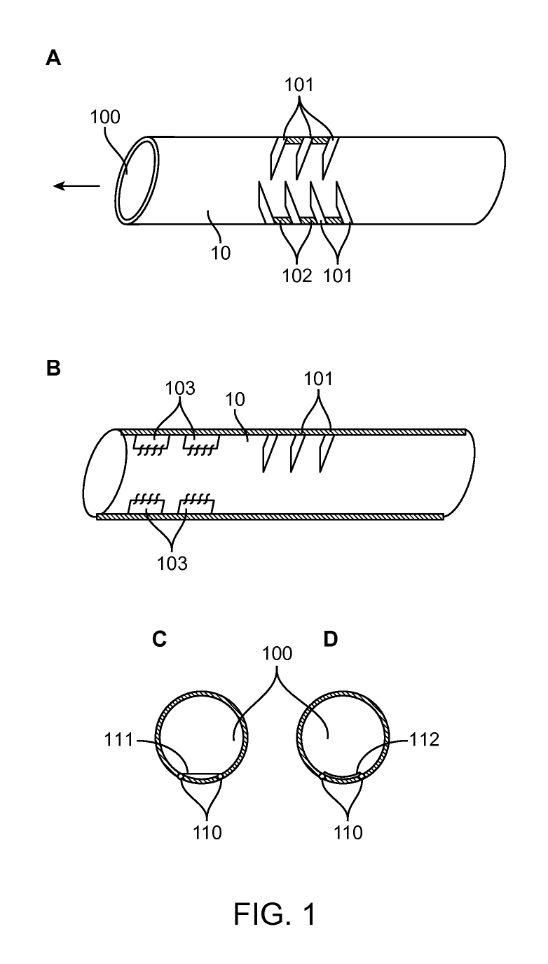

The device described herein comprises three main elements. As shown in FIG. 1, the first main element is, one or more sensors 103 either placed or imbedded in either a plastic catheter (including catheters for IV administration, peripherally inserted central catheters 10 (PICC), or central venous line catheters) or a stainless steel needle 10 (or needle 10 of another material) or combination of the two. The catheter or needle will have a “distal” end, which is the end inserted in the subject, and a “proximal” end, which is the end to which tubing, a syringe, and/or wiring may be connected. A sensor 103 for use in the present device can be an aptasensor, enzyme sensor, antibody sensor, or may use an engineered protein, polymer or biospecific element. The readout of this sensor may be via an ion-sensitive field-effect transistor (“ISFET”), impedimetric, amperometric or other electrochemical method, a micromechanical or other sensor readout modality. The sensor(s) may be located at or near the distal end of the catheter or needle.

The second main element, as shown in FIG. 1, is a plurality of exclusionary slits 101 in the catheter, needle, or combination, upstream of the sensor(s), and angled so that fluid flows in through the slits and down past the sensor(s) 103 in the direction of the arrow. The size of these slits 101 will depend on the specific molecule(s) to be sensed. For example, if a small organic molecule (˜500 Da) is the target molecule, or several are the targets, then the slits can be small enough to exclude all proteins and cells. If a protein is the target, then slit sizes can be adjusted to exclude cells, and possibly larger proteins. Slit dimensions, orientation, and placement can be altered to optimize the flow through the lumen 100 of the catheter 10. Slit design may facilitate reducing flow past the sensing element(s) 103 in the event that the kinetics of the sensor require incubation time. Slit design may be altered depending on eventual in vivo location of a given sensor. For example, the design for a central catheter may be distinct from the design for a peripheral catheter.

The third main element is the wiring of the sensor(s). The wiring 110 may be embedded in the wall of the catheter or needle, or disposed along the inner wall of the catheter or needle, within the lumen. Wiring will allow for signal transduction. Wiring can either be traditional insulated wires, polyimide thin flex, or the like. The use of a flexible wiring connector allows fitting more connections in the catheter or needle.

Optionally, heparin, warfarin, low molecular weight heparin, riveroxiban, or other anticoagulant drugs can be impregnated in parts of the catheter 102 to reduce the risk of occlusion of the slits by clotting factors and/or proteins. Similarly, catheter material can be impregnated with antibiotics, such as rifampicin, clindamycin, aminoglycosides, or tetracycline, to reduce the risk of infection. Other options such as a mechanical cleaning device or delivery of current could be used to unclog the slits 101.

An additional technique that may be used to prevent occlusion of the slits and exclude large molecules that might foul the biosensor is the placement of microfilter membrane over the slits 101. Semipermeable membranes and microfilters can further limit the size of molecules that enter the catheter beyond the size of the slits. For example, 5 μm filter (has 5 μm-sized holes) can exclude 8-10 μm red blood cells from entering into the catheter. The semipermeable membranes used on microdialysis probes are examples of materials that could be used to modify the exclusionary properties of the slits and membranes.

Also disclosed herein is a method for detection of small molecules using the device described above.

Injection of a very small amount of intravenous (“IV”) fluid can be used to act as a fluid barrier to prevent blood cells and proteins from fouling the biosensor surface. Specifically, IV fluid flowing through the lumen 100 over the sensor(s) 103 at a slow rate (as low as about 0.25 mL/hour to about 5 mL/hour)) may be used to prevent the buildup of blood-borne biofouling agents. Fluid flow around the sensor(s) 103 would still allow for small molecules like drug, such as doxorubicin or aminoglycosides, to diffuse to the surface of the sensor. Similarly, small proteins could diffuse to the surface. Other small molecules (and other small proteins) would also diffuse to the sensor, but are unlikely to cause biofouling or nonspecific signal. The rate of IV fluid delivery can to influence the rate of diffusion and size of molecules allowed to diffuse to the sensor surface.

The method described herein can be used either with or without the mechanical protection of a catheter around the sensor. Fluid flow around the sensor can be adjusted such that the fluid acts as the only barrier between the sensor and the external environment. By modulating speed of fluid flow around the sensor, the sensor can be refreshed in the event that aptamer/enzyme/antibody kinetics do not allow for rapid enough equilibration.

IV fluid may be injected either through the catheter that holds the sensor (catheter has a number of slits to allow influx of target molecule (see above)) or IV fluid is delivered directly next to a wire-like sensor so that fluid flows along the wire, encasing it. In addition, IV fluid may be designed for improved sensor function (i.e., ion concentrations, pH, or other common additions such as glucose, within clinically accepted guidelines and commonly used IV solutions). While some IV fluids may demonstrate preferable sensor performance, each sensor will be characterized for the range of clinically used fluids.

The device may further comprise a null electrode sensor. The null electrode sensor is created by another conductor in the catheter or needle that is not treated with the sensor recognition element. This electrode will serve to calibrate the functioning biosensor for any degradation or change in baseline signal that may occur in vivo, as a result of a physiological change (such as blood pH shift) or biofouling. The null sensor may be covered in a similar bio-recognition material that is not sensitive to the target (or any molecule found in blood), but that indicates the baseline of a 0 M concentration signal for the target-binding sensor. It may also be bare or have a different coating that still serves as an indicator of behavior of the sensor. A null electrode may be employed to indicate any fluctuation in signal associated with changes in composition of the IV fluid being delivered and/or any changes in the baseline signal due to minor degradation caused by shear forces, biofouling, or other sensor degradation. An alternative method for monitoring changes in signal baseline comprises applying an electrochemical measurement method to the sensor electrode that is insensitive to changes in target molecule concentration. For example, in square wave voltammetry testing, some frequencies demonstrate sensitivity to changes in concentration, whereas others do not.

Modulation of fluid flow may be used to “refresh” the sensor in the event that sensor does not release the target molecule well unless in a target-free solution (i.e., by speeding up fluid flow so that target diffusion is reduced during “refresh” periods).

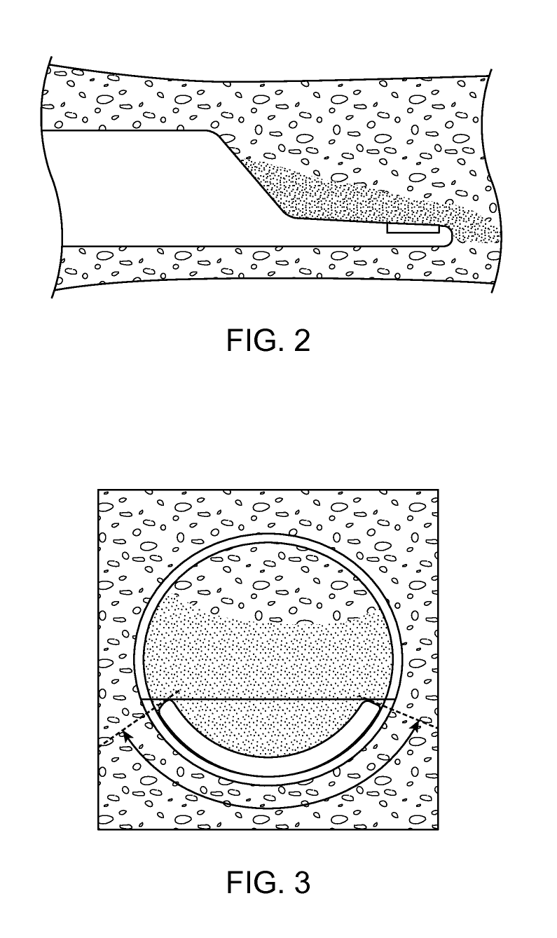

The design of a catheter housing the sensor may be tuned and optimized in order to introduce a boundary (sheath) layer of buffer that will be immediately adjacent to the sensor element, preventing biofouling by cells and large proteins and other molecules, while allowing the smaller analytes of interest to diffuse to the sensor. For example, the catheter may be gradually tapered using a variety of profiles (an example of which is shown in FIG. 2) that result in a thin layer of laminar flow out the distal opening of the catheter. Such a design may be enhanced by features such as guides or internal features, reductions and enhancements in inner tube diameter, which are known to aid in the transition from turbulent to laminar flow, or to change the cross sectional profile of a laminar flow stream. The design of the exclusions slits described elsewhere might also be optimized to introduce desired characteristics (thin laminar flow).

The use of a buffer fluid boundary layer to reduce the effects of biofouling on a biosensor relies on the careful control of flow conditions such as flow rate, pressure, and the degree of turbulence. Several design features in the subject invention are incorporated to exert control over these parameters. A miniature MEMS (micro-electromechanical systems) regulator can be placed in-line with the flow, in order to control the flow rate through the device. Likewise a restrictor or flow orifice may be used for the same purpose. The degree of turbulence (less turbulent, or more laminar, flow is desired for effective diffusion control) in the device may be controlled not only by setting the flow rate to appropriate levels, but also by incorporating features into the flow channel that are specifically designed to produce laminar flow. Examples include converging and diverging flow areas, micro-structured surface features incorporated into the lumen sidewall, and bundles of parallel tubes, honeycomb structures, meshes and nozzles. Other features of the subject invention which are incorporated in order to control aspects of the desired flow include slots, slits, or one or more holes in the tube sidewall, allowing control of the way in which blood flow is introduced into the laminar buffer fluid stream. A multi-lumen tube could also be used where blood is admitted into the inner lumen (by strategically placed slits/slots/holes on the wall of the tube) and a sheath flow of buffer is then formed around the blood. This sheath flow of buffer over the sensor reduces biofouling because the blood will have to diffuse through the sheath flow and make it way to the sensor.

A catheter or needle according to the present invention may have a single lumen, or two or more lumens. In multiple-lumen embodiments, the lumens may be concentric, or may divide the lumen into sections. For example, one such double lumen design divides a circular lumen into two half circles. A double-lumen design may be used, for example, to separate sensors that are measuring an analyte that is being delivered in the IV fluid. For example, in order to measure blood stream glucose accurately, and prevent the signal from being affected by the concentration of glucose in the IV fluid, the sensors may be isolated from the glucose IV fluid by being in a separate lumen. Non-glucose IV fluid would then be required to flow through the sensor-containing lumen. Additionally, a double semi-circle lumen catheter may be used to control the fluid dynamics of blood entering the catheter to promote improved laminar flow, or to slow down the flow rate sufficiently to detect the target molecule. In this embodiment, slits may be both on the outside of the catheter, as well as in the wall within the catheter that separates the two lumens.

An alternative double-lumen embodiment of this device may include two or more concentric lumens. Similarly to above, such concentric lumens could be used to separate a sensor from IV fluid containing the analyte or to further engineer the fluid dynamics of the system to promote laminar flow of blood next to the sensor. In an embodiment with concentric lumens, the inner and outer lumens may be defined by different materials; so, for example, the outer lumen may be a flexible acrylic catheter, while the inner lumen may end in a rigid metallic tip. Alternatively, the outer lumen may include a beveled tip configured to penetrate the skin and vasculature of a subject, while the inner lumen is defined by a flexible material throughout its length.

A concentric double lumen design may be used to protect the sensor during implantation into the body. The outside or leading lumen would take the brunt of the forces during implantation, leaving the inner lumen (and sensors) undisturbed. A concentric-lumen embodiment may include a traditional IV catheter that includes a plastic sheath lumen around a metal needle. In such an embodiment, the outside lumen is the plastic sheath (which remains in the body). The metal needle protrudes past the plastic, and so is used to penetrate the tissue. After placement in the vein, the inner metal needle is removed, leaving the plastic only. In such an embodiment the sensors would be included in the plastic (outer lumen).

In some embodiments of this device, exposure of sensors may be controlled to either 1) protect the sensor during deployment into the body, or 2) to prolong the sensing ability of the device by sequentially exposing sensors. Methods for covering the sensors may include covering the sensor(s) with a degradable material which is applied to the sensor prior to implantation. The degradable material can be applied in a manner such that the degradable material will erode or degrade away, exposing the sensor, in response to the shearing force of the IV fluid or by other factors such as in vivo pH or enzyme degradation. Accordingly, the degradable material can be a hydrogel, polymer, peptide-based hydrogel, natural product such as chitosan, or other material. In a multi-sensor embodiment, varying thicknesses of degradable material can be applied to different sensors in order to exposing them in a predetermined sequence in order to extend the time in which data may be collected beyond the lifetime of a single sensor.

In an alternative embodiment, the sensor(s) can be recessed into the catheter or needle, and a covering comprising degradable material, or a thin metal film, is applied over the opening. This covering may be removed by applying a small current to the edges of the opening to dissipate the material, thus ensuring that sensing (and concomitant degradation of the sensor) does not occur immediately upon implantation, but instead can be delayed until a later time, e.g., during critical periods of patient care.

In some embodiments of the invention, active electronics may be incorporated into the device. For example, a hermetically sealed ASIC (application-specific integrated circuit) or multi-chip module may be integrated in order to drive and read out the biosensor signal. A potentiostat ASIC or multi chip module may be incorporated to drive and read out an electrochemical biosensor. Similarly, an ASIC could be used to perform signal processing functions such as digitization, self-test, offset compensation and calibration. In some embodiments it may be useful for data transmission to be wireless, in which case integrated electronics to transmit data to a nearby or distant receiver may be incorporated. In all cases, electronics may be integrated proximally to the flow device, or it may be packaged more distally with appropriate wiring interconnect between the biosensor and other elements of the device and the electronics unit.

In the embodiment as illustrated in FIG. 3, diffusion of target molecules from blood into the buffer and eventually reaching the sensor is required. The needle has an extended tip with the biosensor patch at a specific distance from the main body of the needle. This distance may be engineered to be sufficient for enough concentration of target molecules to reach the sensor while blood flows through the blood vessel naturally and while buffer solution flows through the needle tip with a controlled volume flow rate.

Overall catheter design and fluid dynamic design can be varied for various types of sensors. This design will be dependent on analyte properties such as size, diffusivity, and physiological concentrations). Design will also be dependent on the binding characteristics of the biorecognition element used to create the biosensor. For example, an aptamer with a slow Kₒₙ rate may require a slower moving sheath fluid layer in order to adequately bind to the target.

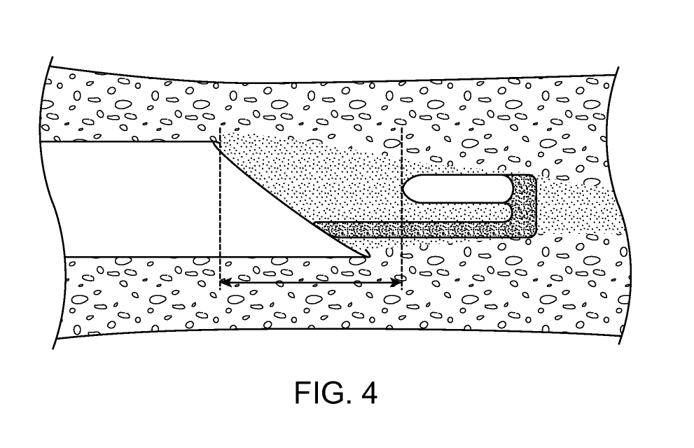

In the embodiment shown in FIG. 4, the sensor is placed on the tip of an inverted hook like structure, which can be retracted or advanced through the needle tip. The sensor may be fully retracted during insertion of needle into the blood vessel and can be advanced to a designed distance once the needle is fully inside the blood vessel. Buffer solution may flow through at a designed and controlled volume flow rate. The amount of advancement of the sensor structure and buffer flow rate will specify the concentration of target molecule that reaches the sensor. Adjustment for blood flow rate and type of sensor is possible through advancement/retraction of sensor structure.

Also described herein is a catheter-based system for placing the sensor in the subject's blood stream. This system may include an electrical connection that can plug into proprietary catheter tubing, which would convey signal out to a readout device or potentiostat. The proprietary catheter tubing may include electrical connections that connect the sensor to a readout box located near other catheter-related medical equipment. Additionally, the proprietary catheter tubing would include connections that allow for electrical contact to the sensor.

As shown in FIGS. 1C and 1D, the sensor may be a gold surface, such as a wire 111 or a microfabricated silicon piece with a gold site 112, that is covered in a polymer layer (or other layer for DNA/RNA attachment), and an aptamer layer that interfaces with the bloodstream but is not necessarily covered in polymer. Aptamers can be selected to be specific for the drug/dye/marker that is being used to calculate blood volume, if that is the use application for a particular sensor. Aptamers can also be selected for drugs or biomarkers for Therapeutic Drug Monitoring or as a diagnostic/theranostic tool with respect to biomarker monitoring.

The system may comprise multiple sensors with coverings, so that the same catheter may be used for multiple detections over the course of a surgery, recovery, blood infusions, etc.

An array of electrodes may be used to continuously monitor a panel. Different products/panels may be applicable in different clinical settings.

### Method for the Embedding of Multiple Electrodes Inside an Intravenous Catheter.

The form and fit of the device is intended to be similar to existing catheters so that usability is not a challenge. In one embodiment, the catheter is intended for both adult and neonatal care, so the catheter dimensions may range from 28 or 24 gauge (for infants) up to 14 gauge (for adults). For the smallest size required (28 gauge), a typical 28 gauge needed has an ID of 184 μm and a wall thickness of 89 μm. This would mean that the conductors used for this application would need to be at the maximum ˜50 μm in diameter These conductors may be coated in an insulating material that allows for the sensing area to be isolated to the tip of the catheter. The areas not covered by insulation become the sensing sites. The area of conductor that is exposed to allow for sensing may depend on the application (which target molecule, adult versus child patient, etc.).

### Methods for Sensor Fabrication

A sensor for use in the device described herein may be fabricated from a variety of materials. In one embodiment, a sensor is a thin flex-like sensor, which may comprise gold pads on polyimide. Such construction permits the user to roll up the sensors and insert them into a catheter or needle.

A sensor for use in the device described herein need not be microfabricated. Instead, a wire, for example a gold wire, may be embedded into a catheter through a molding process This permits constructing a device that comprises multiple parallel wires for multiple sensors (which is capable of measuring multiple analytes, or alternatively may be used if multiple sensors are required for an average measurement, or for other purposes.

### Geometry of Sensors.

Where a device as described herein comprises multiple sensors, those sensors may be arranged in different geometries, such as an array of small squares (like probe sites); or long thin parallel lines of exposed gold to reduce variation across sensors due to fluid dynamics.

### Fabrication of the Catheter.

Conventional catheters are made by drawing plastic to form a tube. This tube is then cut to size and the end is shaped by a hot forming process. The formed and cut tube is then assembled into standard medical fittings like a Luer lock.

The fabrication of a catheter according to the present invention can be achieved in several ways. Interconnect and/or biosensing electrodes can be fabricated by a co-molding process along with the plastic catheter. Alternatively, leads and electrodes can be patterned onto a separate insert which can then be integrated or incorporated with the catheter. The catheter can be built photolithographically, with micro-scale integrated interconnect and a very small inner-diameter sealed micro-channel (lumen) on a planar micro-fabricated surface. This device, upon being released, can then be shaped and or be encapsulated to form the finished catheter. Designs include dimensions for needle gauges ranging from 14-28, in order to encompass all clinically relevant sizes.

### Additive Manufacturing.

In an alternative embodiment, the conductors are embedded in the catheter by an additive manufacturing process. For example, the conductors are mounted on a mandrel and plastic is added over the mandrel to get the desired wall thickness. This is an additive step. Once the plastic sets the mandrel is removed and the conductors will be partially embedded in the wall of the catheter. There is a possibility of insulating the entire length of the conductors and then reflowing the insulation to expose only a known length of the conductors if desired.

### Micro Fabrication:

In an alternative embodiment, gold is sputtered on a thin plastic film. Following that the film is rolled over a mandrel and seam welded to form the catheter tube.

### Routing of the Conductors:

Two options for the routing of the conductors include a Wye adapter, and a Luer lock connector. By using a Wye adapter the conductors can be routed into one of the legs of the Wye and that leg is potted with an epoxy. The other leg is connected to the buffer solution. Alternatively, a custom Luer lock connector, as shown in FIG. 6, provides a smaller design profile. The conductors may be routed to pads on the barrel of the Luer lock connector as seen in FIG. 6. The custom female Luer lock connector may have spring loaded pins which make contact with the pads on the barrel when the device is assembled.

### Method of Operation.

At the beginning of surgery, drug infusion/dosing, or ICU admission, a catheter instrumented with aptamer-functionalized sensors can be inserted in the arm of the patient or through a central line or PICC line. This can be the same catheter that is used to deliver drugs and for all other purposes that an IV catheter is used in the OR. The sensor can either be continuously exposed to the blood stream, or have a covering layer that can be removed at the discretion of an operator (who, for one embodiment, may be the anesthesiologist). In the case of blood volume calculation, at the time when it is desired to measure the circulating blood volume, the operator can inject the marker intravenously. This marker can be any molecule that has no undesired pharmacological effects and is quickly cleared from the blood.

The readout may display a plot of instantaneous blood concentration of the marker. This may include the bolus phase and the plateau reached shortly after injection (for example, approximately 15 seconds after). The readout can then also display the calculated circulating blood volume.

In the case of biomarker monitoring, this molecule for measuring blood volume does not apply.

The sensor may be covered, and the cover may be micro-spring loaded and release with applied current to retract covering layer into the catheter.

### Method for Calculating Total Circulating Blood Volume.

For use in calculating blood volume, the sensor may comprise a microwire sensor or microfabricated sensor, functionalized (optionally with the use of an intermediate polymer layer) with an aptamer layer for the specific detection of whatever marker or dye is being use (among many options, some examples include hippuric acid, I¹²⁵-labeled human serum albumin, and iodinated-RISA). In order to determine the total blood volume, a known number of moles (and volume of dye) would be injected into the patient. The dye sensor would capture the concentration of the dye continuously, thus include the concentration when the dye is distributed through the entire circulatory system (several seconds to minutes). The total blood volume can be calculated using the equation: C₁×V₁=C₂×V₂. In other words, if the initial concentration and volume of the dye injected (C₁×V₁) is known, and the sensor measured C₂, then this equation can be solved for the total volume (blood total volume) throughout which the dye is distributed.

### Method for Therapeutic Drug Monitoring (TDM).

This device can also be used for therapeutic drug monitoring (TDM) in cases for which subjects require close monitoring for early doses. TDM is generally performed for drugs which have significant toxicities if overdosing occurs. TDM may also be used to achieve a specific desired dose, taking into account interindividual pharmacokinetic variability, which may have demonstrated improved benefit to the patient.

It should be understood that the preceding is merely a detailed description of various embodiments of this invention and that numerous changes to the disclosed embodiments can be made in accordance with the disclosure herein without departing from the spirit or scope of the invention. The preceding description, therefore, is not meant to limit the scope of the invention. Rather, the scope of the invention is to be determined only by the appended claims and their equivalents.

What is claimed is:

1. A device comprising a catheter or needle comprising a lumen, a proximal opening, a distal opening, a plurality of exclusionary slits, at least one sensor, and wiring connected to the sensor, wherein the wiring is configured to permit an external connection at the proximal end of the device.

2. The device of claim 1, wherein the sensor is mounted to the interior wall of the catheter or needle.

3. The device of claim 1, wherein the sensor is positioned within the lumen.

4. The device of claim 3, wherein the sensor is slidably positioned within the lumen.

5. The device of claim 4, wherein the device is configured to permit the sensor to be positioned outside the lumen following insertion of the catheter or needle into a subject.

6. The device of claim 1, wherein the sensor is an aptasensor, enzyme sensor, or ion-sensitive field-effect transistor sensor.

7. The device of claim 1, wherein the slits are located closer to the proximal opening than the sensor.

8. The device of claim 1, wherein the sensor is located at the distal end of the device.

9. The device of claim 1, wherein the slits are small enough to exclude blood cells and proteins while allowing plasma to pass through.

10. The device of claim 1, wherein the slits are configured to reduce flow of fluid through the lumen past the biosensor.

11. The device of claim 1, wherein the wiring comprises traditional insulated wires, polyimide thin flex, or a combination thereof.

12. The device of claim 1, wherein the catheter or needle further comprises an anticoagulant or an antibiotic.

13. The device of claim 12, wherein the anticoagulant is heparin, warfarin, low molecular weight heparin, or riveroxiban.

14. The device of claim 12, wherein the antibiotic is rifampicin, clindamycin, aminoglycosides, or tetracycline.

15. The device of claim 1, wherein one or more of the slits are covered with a semi-permeable membrane or a microfilter.

16. The device of claim 15, wherein the microfilter excludes particles larger than 5 μm.

17. The device of claim 15, wherein the semi-permeable membrane is a microdialysis membrane.

18. The device of claim 1, further comprising a null electrode sensor.

19. The device of claim 18, wherein the null sensor is bare, or has a coating that differs from the biosensor.

20. The device of claim 1, wherein the catheter is gradually tapered to produce a thin layer of laminar flow through the distal opening.

21. The device of claim 1, wherein the size of the needle or catheter is from 14 gauge to 28 gauge.

22. The device of claim 1, wherein the needle comprises an extended tip, which projects from the distal end of the needle past the distal opening, and wherein the biosensor is located on the extended tip.

23. The device of claim 1, wherein the sensor comprises a microwire sensor or microfabricated sensor, functionalized with an aptamer layer.

24. The device of claim 23, wherein the sensor further comprises an intermediate polymer layer.

25. The device of claim 1, wherein the sensor comprises a gold surface, a polymer layer covering the gold surface, and an aptamer layer.

26. The device of claim 1, wherein the catheter comprises more than one lumen

27. The device of claim 1, wherein the sensor comprises at least one conductor.

28. The device of claim 27, wherein the conductor is no more than about 50 μm in diameter.

29. The device of claim 27, wherein the conductor is coated in an insulating material, except for an exposed sensing area at the distal end of the conductor.

30. The device of claim 27, further comprising a Wye adapter or Luer lock connector.

31. The device of claim 30, wherein the Wye adapter comprises two legs, the conductor is routed into one of the legs of the Wye adapter, and the other leg of the Wye adapter is connected to reservoir of a buffer solution.

32. The device of claim 32, wherein the Luer lock connector comprises a pad exposed on the barrel of the connector for each conductor, and to which the conductor is routed.

33. The device of claim 30, further comprising a female Luer lock connector which comprises spring-loaded pins which make contact with the pads on the barrel when the device is assembled.

34. The device of claim 1, wherein the sensor is covered.

35. The device of claim 33, wherein the cover is retractable.

36. The device of claim 35, wherein the cover is configured to retract into the catheter upon the application of electrical current.

37. The device of claim 1, wherein the sensor is a thin flex-like sensor configured such in a manner that permits it to be rolled up and inserted into the catheter or needle.

38. The device of claim 37, wherein the sensor comprises one or more gold pads on polyimide.

39. A method comprising inserting a device according to claim 1 into a subject.

40. The method of claim 39, further comprising injecting or infusing intravenous (“IV”) fluid into the device in an amount sufficient to act as a fluid barrier to prevent blood cells and proteins from fouling the biosensor surface.

41. The method of claim 40, wherein IV fluid flows over the biosensor at a rate of about 0.25 mL/hour to about 5 mL/hour.

42. The method of claim 40, further comprising the step of adjusting the flow of IV fluid to refresh the sensor.

43. A method of calculating total circulating blood volume in a subject, comprising
inserting a device according to claim 1 into a blood vessel of the subject;
intravenously injecting a marker into the bloodstream of the subject, wherein the marker is a molecule that has no undesired pharmacological effects and is quickly cleared from the blood; and
measuring the instantaneous blood concentration of the marker.
